(19) United States
(12) Patent Application Publication
(10) Pub. No.: US 2023/0162846 A1
(43) Pub. Date: May 25, 2023

(54) SYSTEMS AND METHODS FOR PREDICTING HEALTHCARE PROVIDER SPECIALTIES

(71) Applicant: Codoxo, Inc., Atlanta, GA (US)

(72) Inventors: Adam Floyd Hannon, Loganville, GA (US); Saurav Kumar Subedi, Snellville, GA (US); Bridget Elise Morales, Snellville, GA (US); Prasoon Saurabh, Alpharetta, GA (US)

(21) Appl. No.: 18/056,593

(22) Filed: Nov. 17, 2022

Related U.S. Application Data
(60) Provisional application No. 63/264,318, filed on Nov. 19, 2021.

(51) Int. Cl. G16H 40/20; G06Q 40/08; G06N 5/02

(52) U.S. Cl. CPC G16H 40/20; G06Q 40/08; G06N 5/022

(57) ABSTRACT

Methods and systems are provided for assessing a query healthcare claim for fraud, waste, or abuse. An example computer-implemented method of generating a predictive model for predicting healthcare provider specialties involves operating at least one processor to receive historical healthcare claim data. Each healthcare claim can include a claim code, a healthcare provider, and a disclosed specialty. The method further involves operating the at least one processor to generate a code utilization profile for each healthcare provider based on the historical healthcare claim data; receive registry data comprising registry specialties for each healthcare provider; select a training dataset comprising the code utilization profiles and corresponding registry specialties; and train the predictive model with the training dataset to predict a healthcare provider specialty for a healthcare claim.

### CROSS-REFERENCE TO RELATED APPLICATIONS

This application claims priority to U.S. Provisional Patent Application No. 63/264,318 filed Nov. 19, 2021 and titled “SYSTEMS AND METHODS FOR PREDICTING HEALTHCARE PROVIDER SPECIALTIES”, the contents of which are incorporated herein by reference for all purposes.

### FIELD

The described embodiments relate to systems and methods for detecting healthcare fraud, waste, or abuse, and in particular, system and methods for predicting healthcare provider specialties.

### BACKGROUND

Healthcare fraud, waste, and abuse causes significant financial loss in the healthcare system. The detection of healthcare fraud, waste, and abuse is typically based on behavioral analytics of a subject entity compared to the subject entity's peers. The subject entity can be, example, a provider, a healthcare claim, or a patient. A subject entity having anomalous behavior compared to its respective peer group can be further investigated.

### SUMMARY

The various embodiments described herein generally relate to methods (and associated systems configured to implement the methods) for assessing a query healthcare claim for fraud, waste, or abuse. The disclosed methods and systems can relate to predicting healthcare provider specialties.

An example computer-implemented method of generating a predictive model for predicting healthcare provider specialties involves operating at least one processor to receive historical healthcare claim data. Each healthcare claim can include a claim code, a healthcare provider, and a disclosed specialty. The method further involves operating the at least one processor to generate a code utilization profile for each healthcare provider based on the historical healthcare claim data; receive registry data comprising registry specialties for each healthcare provider; select a training dataset comprising the code utilization profiles and corresponding registry specialties; and train the predictive model with the training dataset to predict a healthcare provider specialty for a healthcare claim.

In at least one embodiment, operating the at least one processor to select a training dataset comprising the code utilization profiles and corresponding registry specialties can involve operating the at least one processor to, for each healthcare provider: identify a registry specialty of the registry data for the healthcare provider; generate a specialty correspondence indicator representative of a correspondence between the registry specialty of the registry data and the disclosed specialty of the historical healthcare claim data for the healthcare provider; and determine whether to include, in the training dataset, the code utilization profile and corresponding registry specialty for the healthcare provider based on the specialty correspondence indicator.

In at least one embodiment, operating the at least one processor to generate a specialty correspondence indicator can be based on one or more natural language processing fuzzy algorithms.

In at least one embodiment, operating the at least one processor to generate a specialty correspondence indicator representative of a correspondence between the registry specialty of the registry data and the disclosed specialty of the historical healthcare claim data for the healthcare provider can involve operating the at least one processor to generate at least one preliminary specialty correspondence indicator for the registry specialty and the disclosed specialty; and obtain the specialty correspondence indicator based on the at least one preliminary specialty correspondence indicator.

In at least one embodiment, the at least one preliminary specialty correspondence indicator can include a plurality of preliminary specialty correspondence indicators; and the specialty correspondence indicator can be an average of the plurality of preliminary specialty correspondence indicators.

In at least one embodiment, operating the at least one processor to determine whether to include, in the training dataset, the code utilization profile and corresponding registry specialty for the healthcare provider based on the specialty correspondence indicator can involve operating the at least one processor to exclude, from the training dataset, the code utilization profile and corresponding registry specialty for the healthcare provider if: the specialty correspondence indicator is less than a pre-determined threshold value for the specialty correspondence indicator; or one or more preliminary specialty correspondence indicators of the plurality of preliminary specialty correspondence indicators is less than a pre-determined threshold value for that preliminary specialty correspondence indicator.

In at least one embodiment, the at least one preliminary specialty correspondence indicator can include at least one of: a partial score, a token score, or a weighted score. The partial score can be based on one or more abbreviations in the registry specialty or one or more abbreviations in the disclosed specialty. The token score can be based on at least one token word of the registry specialty or at least one token word of the disclosed specialty. The weighted score being based on a length of the registry specialty and a length of the disclosed specialty.

In at least one embodiment, the token score can be based on a ratio of a set of token words of the registry specialty and a set of token words of the disclosed specialty.

In at least one embodiment, the weighted score can be the partial score if the length of the registry specialty is significantly longer or shorter than the length of the disclosed specialty.

In at least one embodiment, operating the at least one processor to generate a code utilization profile for each healthcare provider based on the historical healthcare claim data can involve operating the at least one processor to, for each healthcare provider: identify healthcare claims corresponding to the healthcare provider; determine a total number of healthcare claims corresponding to the healthcare provider; for each healthcare claim code, determine a number of healthcare claims corresponding to the healthcare provider; and for each healthcare claim code, determine a utilization percentage based on the number of healthcare claims corresponding to the healthcare provider for the healthcare claim code to the total number of healthcare claims corresponding to the healthcare provider.

In at least one embodiment, operating the at least one processor to select a training dataset comprising the code utilization profiles and corresponding registry specialty can involve operating the at least one processor to: for each code utilization profile and corresponding registry specialty, determine a volume size of the healthcare provider, the volume size being one of small, average, or large; if the healthcare provider is one of small or large volume size, exclude the code utilization profile and corresponding registry specialty from the training dataset; and if the healthcare provider is average, include the code utilization profile and corresponding registry specialty in the training dataset.

In at least one embodiment, the healthcare provider volume size being small, average or large can be based on at least one of a number of healthcare claims associated with the healthcare provider or a number of patients having healthcare claims associated with the healthcare provider.

In at least one embodiment, the healthcare provider specialty can be based on a taxonomy different from a taxonomy of the disclosed specialty and a taxonomy of the registry specialty.

In at least one embodiment, the taxonomy of the healthcare provider specialty can include a classification that corresponds to a plurality of classifications from the taxonomy of the disclosed specialty or the taxonomy of the registry specialty.

In at least one embodiment, operating the at least one processor to train the predictive model with the training dataset to predict a healthcare provider specialty for a healthcare claim can involve operating the at least one processor to reduce the training dataset dimensionality.

In at least one embodiment, operating the at least one processor to reduce the training dataset dimensionality can involve operating the at least one processor to bicluster the training dataset.

In at least one embodiment, operating the at least one processor to bicluster the training dataset can involve operating the at least one processor to assign each healthcare claim to at least one of a claim type cluster grouping and a business code cluster grouping.

In at least one embodiment, operating the at least one processor to reduce the training dataset dimensionality can involve operating the at least one processor to apply recursive feature elimination to the training dataset.

In at least one embodiment, the method can involve operating the at least one processor to compare the healthcare provider specialty predicted by the predictive model to one or more pre-determined business rules.

In another broad aspect, an example system for generating a predictive model for predicting healthcare provider specialties is disclosed herein. The system includes at least one processor configured to receive historical healthcare claim data. Each healthcare claim can include a claim code, a healthcare provider, and a disclosed specialty. The at least one processor is further configured to generate a code utilization profile for each healthcare provider based on the historical healthcare claim data; receive registry data comprising registry specialties for each healthcare provider; select a training dataset comprising the code utilization profiles and corresponding registry specialties; and train the predictive model with the training dataset to predict a healthcare provider specialty for a healthcare claim.

In at least one embodiment, the at least one processor configured to select a training dataset comprising the code utilization profiles and corresponding registry specialties can include the at least one processor configured to, for each healthcare provider: identify a registry specialty of the registry data for the healthcare provider; generate a specialty correspondence indicator representative of a correspondence between the registry specialty of the registry data and the disclosed specialty of the historical healthcare claim data for the healthcare provider; and determine whether to include, in the training dataset, the code utilization profile and corresponding registry specialty for the healthcare provider based on the specialty correspondence indicator.

In at least one embodiment, the at least one processor configured to generate a specialty correspondence indicator can be based on one or more natural language processing fuzzy algorithms.

In at least one embodiment, the at least one processor configured to generate a specialty correspondence indicator representative of a correspondence between the registry specialty of the registry data and the disclosed specialty of the historical healthcare claim data for the healthcare provider can include the at least one processor being configured to: generate at least one preliminary specialty correspondence indicator for the registry specialty and the disclosed specialty; and obtain the specialty correspondence indicator based on the at least one preliminary specialty correspondence indicator.

In at least one embodiment, the at least one preliminary specialty correspondence indicator can include a plurality of preliminary specialty correspondence indicators; and the specialty correspondence indicator can be an average of the plurality of preliminary specialty correspondence indicators.

In at least one embodiment, the at least one processor configured to determine whether to include, in the training dataset, the code utilization profile and corresponding registry specialty for the healthcare provider based on the specialty correspondence indicator can include the at least one processor configured to exclude, from the training dataset, the code utilization profile and corresponding registry specialty for the healthcare provider if: the specialty correspondence indicator is less than a pre-determined threshold value for the specialty correspondence indicator; or one or more preliminary specialty correspondence indicators of the plurality of preliminary specialty correspondence indicators is less than a pre-determined threshold value for that preliminary specialty correspondence indicator.

In at least one embodiment, the at least one preliminary specialty correspondence indicator can include at least one of: a partial score, a token score, or a weighted score. The partial score can be based on one or more abbreviations in the registry specialty or one or more abbreviations in the disclosed specialty. The token score can be based on at least one token word of the registry specialty or at least one token word of the disclosed specialty. The weighted score being based on a length of the registry specialty and a length of the disclosed specialty.

In at least one embodiment, the token score can be based on a ratio of a set of token words of the registry specialty and a set of token words of the disclosed specialty.

In at least one embodiment, the weighted score can be the partial score if the length of the registry specialty is significantly longer or shorter than the length of the disclosed specialty.

In at least one embodiment, the at least one processor configured to generate a code utilization profile for each healthcare provider based on the historical healthcare claim data can include the at least one processor configured to, for each healthcare provider: identify healthcare claims corresponding to the healthcare provider; determine a total number of healthcare claims corresponding to the healthcare provider; for each healthcare claim code, determine a number of healthcare claims corresponding to the healthcare provider; and for each healthcare claim code, determine a utilization percentage based on the number of healthcare claims corresponding to the healthcare provider for the healthcare claim code to the total number of healthcare claims corresponding to the healthcare provider.

In at least one embodiment, the at least one processor configured to select a training dataset comprising the code utilization profiles and corresponding registry specialty can include the at least one processor configured to: for each code utilization profile and corresponding registry specialty, determine a volume size of the healthcare provider, the volume size being one of small, average, or large; if the healthcare provider is one of small or large volume size, exclude the code utilization profile and corresponding registry specialty from the training dataset; and if the healthcare provider is average, include the code utilization profile and corresponding registry specialty in the training dataset.

In at least one embodiment, the healthcare provider volume size being small, average or large can be based on at least one of a number of healthcare claims associated with the healthcare provider or a number of patients having healthcare claims associated with the healthcare provider.

In at least one embodiment, the healthcare provider specialty can be based on a taxonomy different from a taxonomy of the disclosed specialty and a taxonomy of the registry specialty.

In at least one embodiment, the taxonomy of the healthcare provider specialty can include a classification that corresponds to a plurality of classifications from the taxonomy of the disclosed specialty or the taxonomy of the registry specialty.

In at least one embodiment, the at least one processor configured to train the predictive model with the training dataset to predict a healthcare provider specialty for a healthcare claim can include the at least one processor configured to reduce the training dataset dimensionality.

In at least one embodiment, the at least one processor configured to reduce the training dataset dimensionality can include the at least one processor configured to bicluster the training dataset.

In at least one embodiment, the at least one processor configured to bicluster the training dataset can include the at least one processor configured to assign each healthcare claim to at least one of a claim type cluster grouping and a business code cluster grouping.

In at least one embodiment, the at least one processor configured to reduce the training dataset dimensionality can include the at least one processor configured to apply recursive feature elimination to the training dataset.

In at least one embodiment, the at least one processor can be configured to compare the healthcare provider specialty predicted by the predictive model to one or more pre-determined business rules.

In another broad aspect, an example method of assessing a query healthcare claim for fraud, waste, or abuse is disclosed herein. The method involves operating at least one processor to receive healthcare claim data for a healthcare provider. The healthcare claim data includes at least the query healthcare claim. Each healthcare claim of the healthcare claim data includes a claim code and a disclosed specialty. The method further involves operating the at least one processor to generate a query code utilization profile for the healthcare provider of the query healthcare claim; determine a predicted healthcare provider specialty for the query healthcare claim by applying the query code utilization profile to a predictive model generated for predicting a healthcare provider specialty; determine whether a behavior of the healthcare provider of the query healthcare claim data is anomalous based on the predicted healthcare provider specialty; and assess the query healthcare claim based on the behavior of the healthcare provider.

In another broad aspect, an example system for assessing a query healthcare claim for fraud, waste, or abuse is disclosed herein. The system includes at least one processor configured to receive healthcare claim data for a healthcare provider.

The healthcare claim data includes at least the query healthcare claim. Each healthcare claim of the healthcare claim data includes a claim code and a disclosed specialty. The at least one processor is further configured to generate a query code utilization profile for the healthcare provider of the query healthcare claim; determine a predicted healthcare provider specialty for the query healthcare claim by applying the query code utilization profile to a predictive model generated for predicting a healthcare provider specialty; determine whether a behavior of the healthcare provider of the query healthcare claim data is anomalous based on the predicted healthcare provider specialty; and assess the query healthcare claim based on the behavior of the healthcare provider.

### BRIEF DESCRIPTION OF THE DRAWINGS

Several embodiments will now be described in detail with reference to the drawings, in which:

FIG. 1 is a block diagram of components interacting with an example healthcare fraud detection system, in accordance with an example embodiment;

FIG. 2 is a flowchart of an example embodiment of various methods of generating a predictive model for predicting healthcare provider specialties;

FIG. 3A is an illustration of example historical healthcare claim data, in accordance with an example embodiment;

FIG. 3B is an illustration of example code utilization profiles for the historical healthcare claim data of FIG. 3A, in accordance with an example embodiment;

FIG. 3C is an illustration of example registry data, in accordance with an example embodiment;

FIG. 3D is an illustration of an example training dataset based on the example code utilization profiles of FIG. 3B and the example registry data of FIG. 3C, in accordance with an example embodiment;

FIG. 4 is an illustration of an example healthcare provider specialty prediction, in accordance with an example embodiment;

FIG. 5 is a block diagram of components interacting in the example method of FIG. 2; and

FIG. 6 is a block diagram of components interacting in an example method for assessing a query healthcare claim, in accordance with an example embodiment.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

The various embodiments described herein generally relate to methods (and associated systems configured to implement the methods) of generating a predictive model for predicting healthcare provider specialties. The systems and methods can use artificial intelligence or machine learning methods to train the predictive model.

Healthcare fraud, waste, and abuse detection is typically based on a comparison of the behavior of a subject entity to the behavior of the subject entity's peers. Accordingly, comparison to appropriate peers is critical. However, the definition of the subject entity's peers can be subjective.

Healthcare providers' specialties are recorded in an external registry, such as a National Provider registry. However, information recorded in external registries may be incorrect or obsolete. The time relevance of such external registries may not be reliable.

For example, healthcare providers specialties are also disclosed with healthcare claims and the specialties disclosed with healthcare claims may not be accurate, and further may not align with the specialties recorded in the external registry. As such, reliance on external registries for the subject entity's peers may result in false positives for anomalous behavior.

The systems and methods described herein operate to predict healthcare provider specialties by analyzing code utilization profiles of healthcare claim data. The system can generate a predictive model for predicting healthcare provider specialties. In some embodiments, the system can receive registry data and historical healthcare claim data and generate a code utilization profile for each healthcare provider based on the historical healthcare claim data. The system can also select a training dataset including the code utilization profiles and corresponding registry specialties of the registry data. The system can use artificial intelligence and/or machine learning methods to train the predictive model with the training dataset to predict a healthcare provider specialty for a healthcare claim.

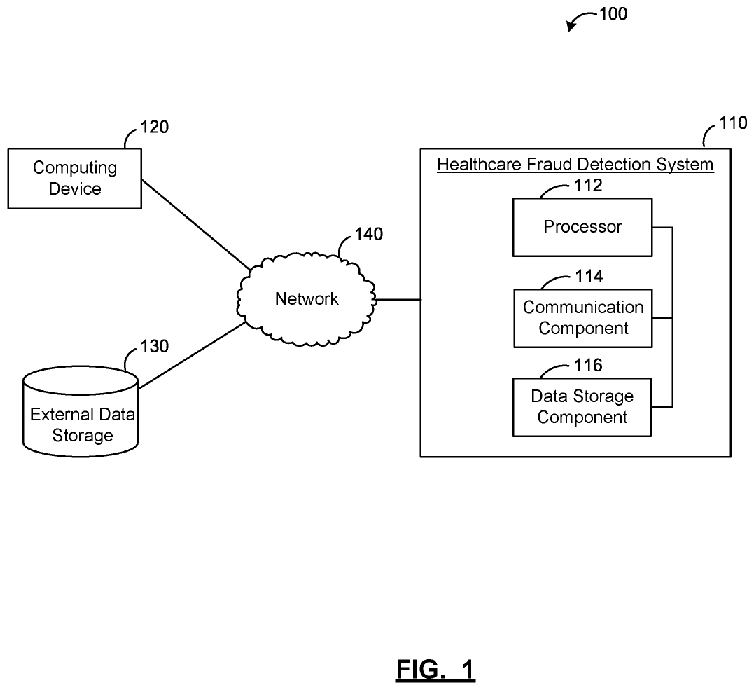

Reference will now be made to FIG. 1, which is a block diagram 100 of components interacting with an example healthcare fraud detection system 110. As shown in FIG. 1, the healthcare fraud detection system 110 is in communication with a computing device 120 and an external data storage 130 via a network 140.

The healthcare fraud detection system 110 includes a processor 112, a communication component 114, and a data storage component 116. The healthcare fraud detection system 110 can be provided on one or more computer servers that may be distributed over a wide geographic area and connected via the network 140.

The healthcare fraud detection system 110 can perform various functions related to the detection of healthcare fraud, waste, or abuse. For example, the healthcare fraud detection system 110 can receive data, such as healthcare claim data, from computing device 120. The healthcare fraud detection system 110 can also access data, such as registry data or medical code definitions, stored in external data storage 130. The healthcare fraud detection system 110 can develop a code utilization profile from healthcare claim data. The healthcare fraud detection system 110 can generate a prediction model for predicting healthcare provider specialties for healthcare claim data.

In some embodiments, each of the processor 112, the communication component 114, and the data storage component 116 can be combined into a fewer number of components or may be separated into further components. The processor 112, the communication component 114, and the data storage component 116 can be implemented in software or hardware, or a combination of software and hardware.

The processor 112 can operate to control the operation of the healthcare fraud detection system 110. The processor 112 can initiate and manage the operations of each of the other components within the healthcare fraud detection system 110. The processor 112 may be any suitable processors, controllers, digital signal processors, or graphics processing units (GPUs) that can provide sufficient processing power depending on the configuration, purposes and requirements of the healthcare fraud detection system 110. In some embodiments, the processor 112 can include more than one processor with each processor being configured to perform different dedicated tasks.

The communication component 114 may include any interface that enables the healthcare fraud detection system 110 to communicate with other devices and systems. In some embodiments, the communication component 114 can include at least one of a serial port, a parallel port or a USB port. The communication component 114 may also include at least one of an Internet, Local Area Network (LAN), Ethernet, Firewire, modem or digital subscriber line connection. Various combinations of these elements may be incorporated within the communication component 114.

For example, the communication component 114 may receive input from various input devices, such as a mouse, a keyboard, a touch screen, a thumbwheel, a track-pad, a track-ball, a card-reader, voice recognition software and the like depending on the requirements and implementation of the healthcare fraud detection system 110.

The data storage component 116 can include RAM, ROM, one or more hard drives, one or more flash drives or some other suitable data storage elements such as disk drives, etc. The data storage component 116 can also include one or more databases (not shown) for storing information relating to, for example, registry data, healthcare claim data, service providers, patients, claim codes, types of treatments and/or procedures, etc.

Similar to the data storage component 116, the external data storage 130 can also include RAM, ROM, one or more hard drives, one or more flash drives or some other suitable data storage elements such as disk drives, etc. In some embodiments, the external storage 130 can be similar to the data storage component 116 but located remotely from the healthcare fraud detection system 110 and accessible via the network 140. The external data storage 130 can also include one or more databases (not shown) for storing information relating to, for example, registry data, healthcare claim data, service providers, patients, claim codes, types of treatments and/or procedures, etc.

The computing device 120 can include any networked device operable to connect to the network 140. A networked device is a device capable of communicating with other devices through a network such as the network 140. A networked device may couple to the network 140 through a wired or wireless connection. Although only one computing device 120 is shown in FIG. 1, it will be understood that more computing devices 120 can connect to the network 140.

The computing device 120 may include at least a processor and memory, and may be an electronic tablet device, a personal computer, workstation, server, portable computer, mobile device, personal digital assistant, laptop, smart phone, WAP phone, an interactive television, video display terminals, gaming consoles, and portable electronic devices or any combination of these.

The network 140 may be any network capable of carrying data, including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX, Ultra-wideband, Bluetooth®), SS7 signaling network, fixed line, local area network, wide area network, and others, including any combination of these, capable of interfacing with, and enabling communication between, the healthcare fraud detection system 110, the external storage 130 and the computing device 120.

It will be understood that some components of FIG. 1, such as components of the healthcare fraud detection management system 110 or the external data storage 130, can be implemented in a cloud computing environment.

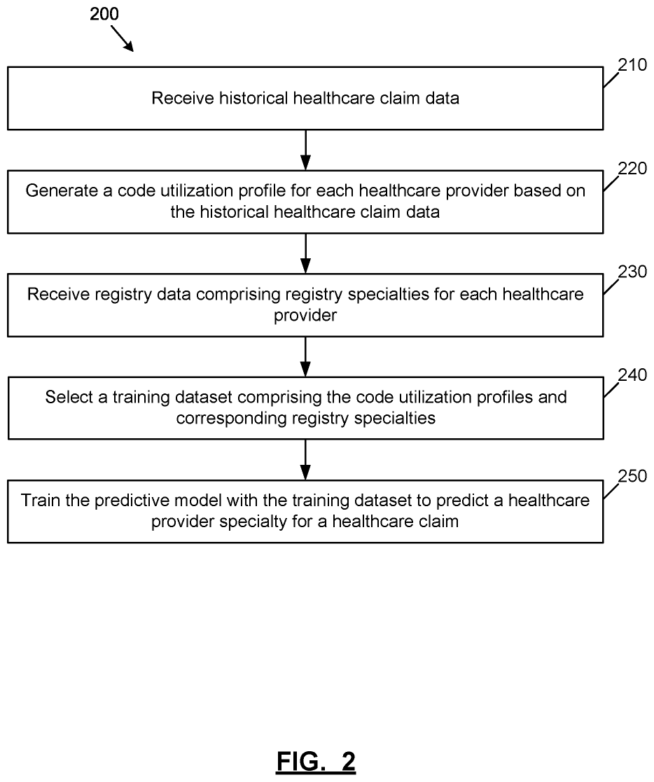

Reference is now made to FIG. 2, which illustrates a flowchart of an example method 200 of generating a predictive model for predicting healthcare provider specialties, in accordance with an example embodiment. A healthcare fraud detection system, such as healthcare fraud detection system 110 having at least one processor 112 can be configured to implement the method 200.

At 210, the processor 112 receives historical healthcare claim data. The historical healthcare claim data can include a plurality of historical healthcare claims. Each historical healthcare claim can include a claim code related to services performed, a healthcare provider who rendered the services, a disclosed specialty of the healthcare provider who rendered the services, and a patient who received the services. The historical healthcare claim data can be received from a computing device, such as computing device 120, or an external data storage, such as external data storage 130.

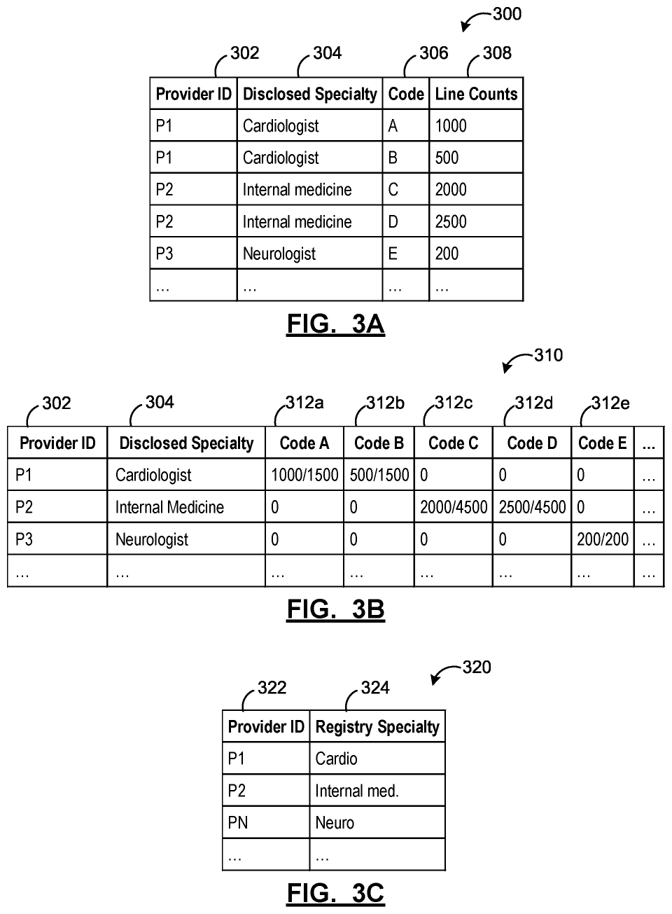

Reference is now made to FIG. 3A, which illustrates example historical healthcare claim data 300, in accordance with an example embodiment. As shown in FIG. 3A, the historical healthcare claim data 300 can include a healthcare provider identifier 302, a specialty disclosed by the healthcare provider 304, healthcare claim codes 306, and line counts 308. Line counts 308 can be a total number of healthcare claims that utilize a healthcare claim code 306. That is, the healthcare claim data 300 shown in FIG. 3A is aggregated data. For example, provider P2 reported code C in 2000 lines of the historical healthcare claims and code D in 2500 line of the historical healthcare claims 300.

The historical healthcare data 300 can be subject to various privacy and security restrictions and/or contractual obligations. Use of aggregated data allows for compliance with such restrictions and obligations.

In order to aggregate the historical healthcare claim data 300, the historical healthcare claim data 300 can be standardized to a common format. For example, the historical healthcare claim data 300 can originate from different sources, having different field names, table structures, and claim codes—including non-standard claim codes. A standard, common format can be adopted.

Returning now to FIG. 2, at 220, the processor 112 generates a code utilization profile for each healthcare provider based on the historical healthcare claim data. To generate a code utilization profile for a healthcare provider, the processor 112 can identify healthcare claims corresponding to the healthcare provider and determine a total number of healthcare claims corresponding to the healthcare provider. For each healthcare claim code, the processor 112 can determine a number of healthcare claims corresponding to the healthcare provider. The processor 112 can, for each healthcare claim code, determine a utilization percentage based on the number of healthcare claims corresponding to the healthcare provider for the healthcare claim code to the total number of healthcare claims corresponding to the healthcare provider.

Reference is now made to FIG. 3B, which illustrates example code utilization profiles 310 for the historical healthcare claim data of FIG. 3A, in accordance with an example embodiment. As shown in FIG. 3B, the code utilization profile for each healthcare provider can include the healthcare provider identifier 302, the specialty disclosed by the healthcare provider 304, and utilization percentages 312a, 312b, 312c, 312d, 312e . . . (herein collectively referred to as the utilization percentages 312) for each healthcare claim code 306. For example, the code utilization for provider P2 is 44.4% (i.e., 2000/4500) for code C and 55.6% (i.e., 2500/4500) for code D.

Returning now to FIG. 2, at 230, the processor 112 receives registry data. The registry data can include registry specialties for each healthcare provider. The registry data can be received from a computing device, such as computing device 120, or an external data storage, such as external data storage 130. The registry data can be stored in a database, such as a National Provider Identifier (NPI) registry that provides the service provider's registration information. The registry specialty recorded in the registry data can be defined in accordance with a particular taxonomy, such as a National Plan and Provider Enumeration System (NPPES).

Reference is now made to FIG. 3C, which illustrates example registry data 320, in accordance with an example embodiment. As shown in FIG. 3C, the registry data can include a healthcare provider identifier 322 and a specialty recorded in the registry 324.

Returning now to FIG. 2, at 240, the processor 112 selects a training dataset. The processor 112 can select a portion of the code utilization profiles generated at 210 and corresponding registry specialties received at 230 to use as the training dataset.

In some embodiments, the processor 112 can use the remaining data (i.e., the portion that is not included in the training dataset) as a validation dataset. For example, 80% of the historical healthcare claim data 300 can be used as the training dataset and the remaining 20% of the historical healthcare claim data 300 can be used as the validation dataset. In some embodiments, class imbalance can also be accounted for. That is, some specialties may not have a comparable number of providers.

In some embodiments, the processor 112 can, for each healthcare provider, identify a registry specialty 324 within the registry data 320 received at 230 for the healthcare provider. The registry specialty 324 of the registry data 320 can be used to validate the disclosed specialty 304 of the historical healthcare claim data 300 for a healthcare provider. The processor 112 can generate a specialty correspondence indicator representative of a correspondence between the registry specialty 324 of the registry data 320 and the disclosed specialty 304 of the historical healthcare claim data 300 for the healthcare provider. For example, a greater value of the specialty correspondence indicator can represent an accurate match between the registry data 320 and the disclosed specialty 304 and a lower value can represent an inaccurate match.

The processor 112 can determine whether to include, in the training dataset 330, the code utilization profile 310 and corresponding registry specialty 324 for the healthcare provider based on the specialty correspondence indicator. For example, the processor 112 can compare the specialty correspondence indicator with a pre-determined threshold value. If the specialty correspondence indicator is greater than or equal to the pre-determined threshold value, the registry data 320 and the disclosed specialty 304 can be considered an accurate match and the code utilization profile 310 and corresponding registry specialty 324 for the healthcare provider can be included in the training dataset 330. If the specialty correspondence indicator is less than the pre-determined threshold value, the registry data 320 and the disclosed specialty 304 may not be considered an accurate match and the code utilization profile 310 and corresponding registry specialty 324 for the healthcare provider can be excluded from the training dataset 330.

The processor 112 can generate a specialty correspondence indicator based on one or more natural language processing (NLP) fuzzy algorithms. In some embodiments, the processor 112 can generate at least one preliminary specialty correspondence indicator for the registry specialty and the disclosed specialty; and obtain the specialty correspondence indicator based on the at least one preliminary specialty correspondence indicator. For example, the processor 112 can generate a plurality of preliminary specialty correspondence indicators and obtain an average of the plurality of preliminary specialty correspondence indicators.

In at least one embodiment, a preliminary specialty correspondence indicator can be a full ratio score between the disclosed specialty 304 and the registry specialty 324. A full ratio score can be determined based on a comparison of string text corresponding to the disclosed specialty 304 and string text corresponding to the registry specialty 324.

In at least one embodiment, a preliminary specialty correspondence indicator can be a partial ratio score, or a partial score, between the disclosed specialty 304 and the registry specialty 324. The partial ratio score can result in a higher value than the full ratio score as the partial ratio score can account for partial strings of the disclosed specialty 304 (e.g., different nomenclature or abbreviations in the disclosed specialty 304) and partial strings of the registry specialty 324 (e.g., different nomenclature or abbreviations in the registry specialty 324). For example, “Physical and Rehab” is an abbreviation of “Physical & Rehabilitation”. A partial ratio of “Physical Rehab” to “Physical & Rehabilitation” can result in a higher score than a full ratio score of “Physical Rehab” to “Physical & Rehabilitation”.

In at least one embodiment, a preliminary specialty correspondence indicator can be a full token set ratio score, or full token set score, between the disclosed specialty 304 and the registry specialty 324. The full token set ratio score can be determined by generating a token word for each word of the disclosed specialty 304 and the registry specialty 324. Duplicate token words of the disclosed specialty 304 and duplicate token words of the registry specialty 324 can be discarded to obtain a set of unique token words for the disclosed specialty 304 and a set of unique token words for the registry specialty 324, respectively. The full token set ratio score can be determined based on a set of unique token words for the disclosed specialty 304 and a set of unique token words for the registry specialty 324. The full token set ratio score can result in a higher value than a full ratio score as the token set ratio score can account for words that share a common token.

For example, a full token set ratio of “Durable Medical Equipment—Oxygen/Respirator” to “Durable Medical Equipment & Medical Supplies” can result in a higher score than a score obtained from a full ratio of “Durable Medical Equipment—Oxygen/Respirator” to “Durable Medical Equipment & Medical Supplies”. That is, the words “oxygen” and “respirator” can share a common token and “respirator” results in a duplicate token word following “oxygen”.

In at least one embodiment, a preliminary specialty correspondence indicator can be a full token sort ratio score, or full token sort score, between the disclosed specialty 304 and the registry specialty 324. The set of unique token words of the disclosed specialty 304 and the set of unique token words of the registry specialty 324 can be sorted to obtain an ordered set of unique token words for the disclosed specialty 304 and an ordered set of unique token words for the registry specialty 324, respectively.

The full token sort ratio score can be determined based on the ordered set of unique token words for the disclosed specialty 304 and an ordered set of unique token words for the registry specialty 324. The full token sort ratio score can result in a higher value than a full token set ratio score because the token sort ratio score can account for similar words being in different orders.

It should be noted that partial strings in the disclosed specialty 304 and the registry specialty 324 can also be accounted for to obtain a partial token set score and a partial token sort score. In at least one embodiment, the processor 112 can determine whether the length of the disclosed specialty 304 is significantly longer than the length of the registry specialty 324, or vice versa. An example threshold for being significantly longer can be about 1.5 times. If the length of either the disclosed specialty 304 or the registry specialty 324 is significantly longer than the other, the processor 112 can determine a partial token set ratio score or a partial token sort ratio score instead of a full token set ratio score or a full token sort ratio score, respectively.

In at least one embodiment, a preliminary specialty correspondence indicator can be a weighted ratio score, or weighted score, between the disclosed specialty 304 and the registry specialty 324. For example, the weighted score can be the greater score of a plurality of scores, such as the full ratio, the full token set ratio, and the full token sort ratio. Furthermore, if the processor 112 determines that the length of the disclosed specialty 304 is signification longer than the length of the registry specialty 324, or vice versa, the weighted score can be the greater score of the full ratio, the partial token set ratio, and the partial token sort ratio. For example, a weighted ratio of “Ob/Gyn” to “Obstetrics & Gynecology” can result in a higher score than a score obtained from a full ratio of “Ob/Gyn” to “Obstetrics & Gynecology”.

In some embodiments, the processor 112 can determine whether to include, in the training dataset 330, the code utilization profile 310 and corresponding registry specialty 324 for the healthcare provider based on the at least one preliminary specialty correspondence indicator. For example, the processor 112 can compare each preliminary specialty correspondence indicator with a pre-determined threshold value for that preliminary specialty correspondence indicator. The pre-determined threshold value for a token set ratio score may be different from the pre-determined threshold value for a partial ratio score.

If a preliminary specialty correspondence indicator is greater than or equal to the pre-determined threshold value, the registry data 320 and the disclosed specialty 304 can be considered an accurate match and the code utilization profile 310 and corresponding registry specialty 324 for the healthcare provider can be included in the training dataset 330. If the preliminary specialty correspondence indicator is less than the pre-determined threshold value, the registry data 320 and the disclosed specialty 304 may not be considered an accurate match and the code utilization profile 310 and corresponding registry specialty 324 for the healthcare provider can be excluded from the training dataset 330.

In some embodiments, the processor 112 can determine whether to include, in the training dataset 330, the code utilization profile 310 and corresponding registry specialty 324 for the healthcare provider based on the specialty correspondence indicator as well as the at least one preliminary specialty correspondence indicator. For example, if any one of the specialty correspondence indicator or the preliminary specialty correspondence indicators are less than a respective pre-determined threshold value, the registry data 320 and the disclosed specialty 304 may not be considered an accurate match and the code utilization profile 310 and corresponding registry specialty 324 for the healthcare provider can be excluded from the training dataset 330. That is, the registry data 320 and the disclosed specialty 304 can only be considered an accurate match and included in the training dataset 330 if the specialty correspondence indicator and each of the at least one preliminary specialty correspondence indicator is greater than the respective pre-determined threshold value.

In some embodiments, the processor 112 can determine whether to include, in the training dataset 330, the code utilization profile 310 and corresponding registry specialty 324 for the healthcare provider based a volume size of the healthcare provider. Low volume and/or extremely large volume healthcare providers can skew the overall distribution. As such, the processor 112 can exclude such healthcare providers from the training dataset 330. In some examples, excluded healthcare providers can represent about 1 to 3% of the top and bottom of the distribution, depending on the healthcare claim type and healthcare provider specialty. Extremely large volume healthcare providers can include, but is not limited to, national laboratories. Healthcare providers with a very small number of patients, or a very small number of claims, or both a very small number of patients and a very small number of claims can be considered low volume healthcare providers. In some embodiments, the processor 112 can determine that only healthcare providers having an average volume size can be included in the training dataset 330.

Reference is now made to FIG. 3D, which illustrates an example training dataset 330, in accordance with an example embodiment. As shown in FIG. 3D, the training dataset 330 can include the code utilization profiles, such as the healthcare provider identifier 302, the disclosed specialty 304, and the utilization percentages 312, and the registry specialty 324. In example training dataset 330, the processor 112 selected providers P1, P2, and P4 to be included in the training dataset 330 and provider P3 to be excluded from the training dataset.

Returning now to FIG. 2, at 250, the processor 112 trains the predictive model with the training dataset selected at 240 to predict a healthcare provider specialty for a healthcare claim. The processor 112 can train the predictive model using artificial intelligence and/or machine learning methods. The healthcare provider specialty predicted by the predictive model can be based on a taxonomy that is different from the taxonomy of the registry specialty 324 and/or the taxonomy of the disclosed specialty 304.

Using a more robust taxonomy for the predictive model can minimize the misclassification rate. A taxonomy with larger groups can be more robust. For example, the taxonomy of the predictive model can include “general medicine” as a specialty. The “general medicine” specialty of the predictive model can encompass specialties such as “internal medicine”, “family medicine”, and “nurse practitioner” of the disclosed specialty 304 or the registry specialty 324. Similarly, “social worker”, “psychiatrist”, and “counselor” specialties in the disclosed specialties 304 or the registry specialties 324 can be combined into a single specialty in the taxonomy of the predictive model. A taxonomy with larger groups can also allow for easier identification of specialties within the larger groups, such as internal medicine and surgery. The taxonomy of the predictive model can be developed through a reiterative process. In some cases, the taxonomy of the predictive model can be manually developed based on the knowledge subject matter experts.

The training dataset 330 can be a high dimensional and sparse dataset. To improve the accuracy of the predictive model, the processor 112 can reduce the dimensionality of the training dataset 330. The dimensionality of the training dataset 330 can be reduced by biclustering the training dataset 330. For example, the processor 112 can cluster the training dataset 330 based on claim type groupings and cluster the claim type groupings by business code groupings. Example claim type groupings can include, but is not limited to, professional, dental, pharmacy, and facility. Example business code groupings for professional claim types can include, but is not limited to, Current Procedural Terminology (CPT) code groups and diagnosis code groups. Example business code groupings for pharmacy claim types can include but is not limited to therapeutic code groups. To cluster the training dataset 330, the processor 112 can assign each healthcare claim to one of the clusters.

To further reduce the dimensionality of the training dataset 330 and thereby enhance signal to noise ratio, the processor 112 can apply recursive feature elimination to the training dataset 330. In particular, the processor 112 can apply recursive feature elimination to the business code groupings. An example of business code groupings are Clinical Classification Software (CCS) groups for CPT codes.

The processor 112 can compare the healthcare provider specialty predicted by the predictive model to one or more pre-determined business rules and enforce the pre-determined business rules on the specialty prediction. The pre-determined business rules can be manually developed based on knowledge of subject matter experts. The pre-determined business rules can relate to, but is not limited to, time behavior, network coverage, geographic coverage. For example, a business rule can relate to the place of service (POS) code location. In particular, a business rule can require that certain healthcare provider specialties are only practiced at a select POS code locations. Accordingly, the processor 112 can identify healthcare claims associated with a particular healthcare provider specialty and POS code location that is not one of the select POS code locations for that healthcare provider specialty.

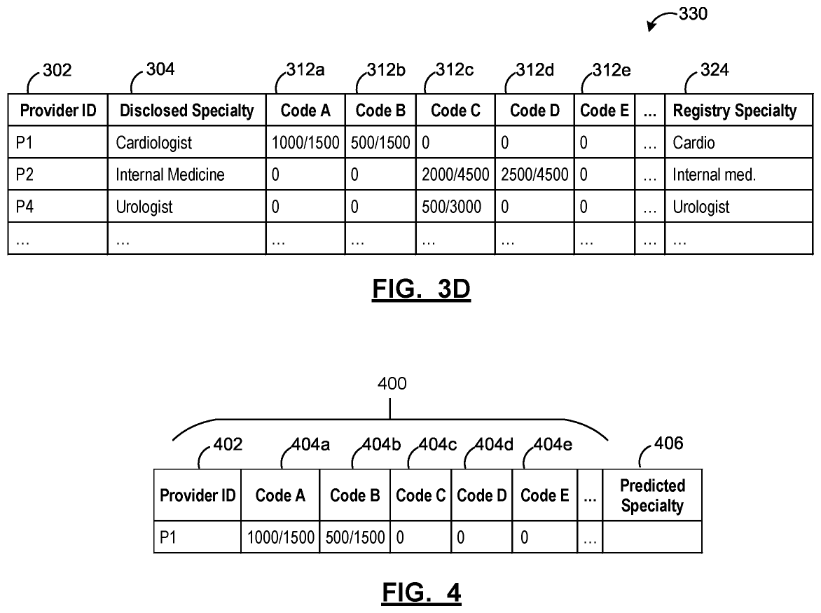

Reference is now made to FIG. 4, which illustrates an example healthcare provider specialty prediction generated by the predictive model, in accordance with an example embodiment. As shown in FIG. 4, the predictive model can receive a query code utilization profile 400 for a healthcare provider 402, including the utilization percentages 404a, 404b, 404c, 404d, 404e . . . (herein collectively referred to as utilization percentages 404) for various healthcare claim codes. The predictive model can return a predicted specialty 406 for the healthcare provider.

In some embodiments, the processor 112 can retrain the predictive model, depending on the business need. For example, the processor 112 can retrain the predictive model on a quarterly or semi-annual basis. The processor 112 can also monitor the performance of the predictive model and automatically retrain the predictive model when a model drift, or a degradation in performance below a pre-determined threshold, is observed.

The predictive model can be trained using multiple algorithms and the best performing model can be identified during validation using metrics such as a precision/recall curve and a ROC-AUC curve.

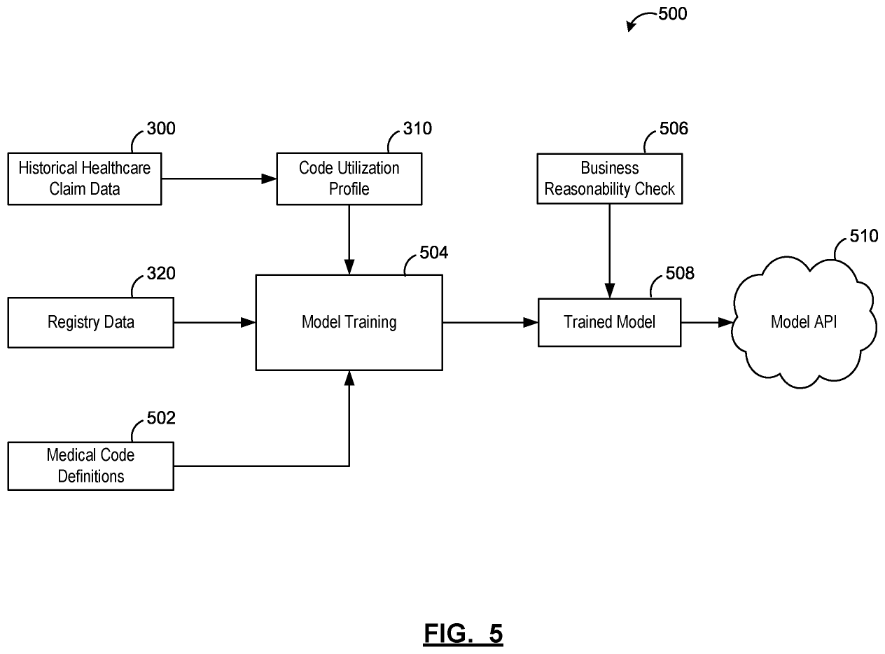

Reference is now made to FIG. 5, which illustrates a block diagram 500 of components interacting in example method 200, in accordance with an example embodiment. The processor 112 receives historical healthcare claim data 300 at 210 and generates code utilization profiles 310 for each healthcare provider at 220. The processor 112 also receives registry data 320 at 230 and compares the code utilization profiles 310 with the registry data 320. The processor 112 can select a training dataset 330 from the historical healthcare claim data 300 and the registry data 320 at 240, based on the size volume of the healthcare providers of the claims data 300 and the correspondence between the disclosed specialties of the claim data 300 and the registry specialties of the registry data 320.

The training dataset 330 can be sparse and have high dimensionality. To reduce the dimensionality, the processor 112 can bicluster the training dataset 330 based on the claim codes used in the historical healthcare claim data 300. For example, the processor 112 can receive medical code definitions 502 from an external data source, such as external data storage 130, and cluster and classify the training dataset 330 based on the medical code definitions 502.

Additional pre-determined business rules can be applied to the healthcare provider specialty as predicted by the predictive model to provide a business reasonability check 506. The pre-determined business rules can relate to, but is not limited to, time behavior, network coverage, geographic coverage. In some embodiments, the predictive model and pre-determined business rules can be implemented as an application programming interface 510. Furthermore, implementation as an application programming interface reduces latency requirements.

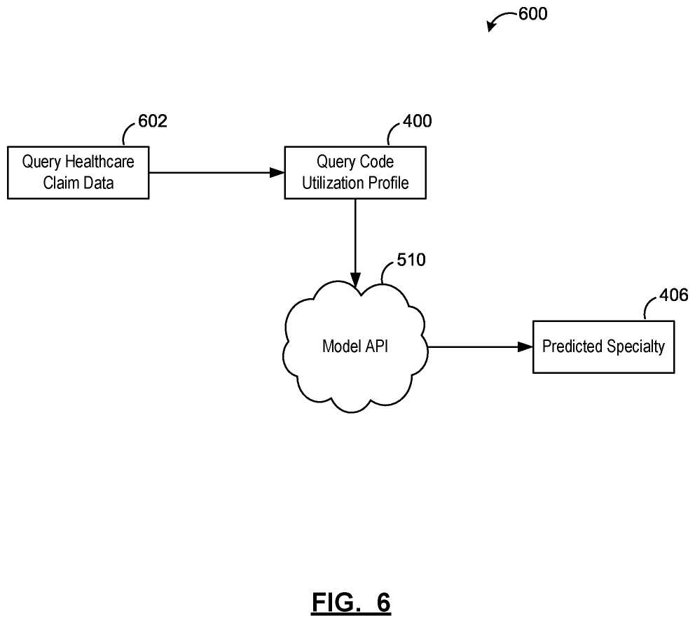

Reference is now made to FIG. 6, which illustrates a block diagram 600 of components interacting in an example method for assessing a query healthcare claim for fraud, waste, or abuse, in accordance with an example embodiment. A healthcare fraud detection system, such as healthcare fraud detection system 110 having at least one processor 112 can be configured to implement the example method for assessing a query healthcare claim.

The processor 112 can receive query healthcare claim data for a healthcare provider. The healthcare claim data can include at least the query healthcare claim 602. Each healthcare claim of the healthcare claim data can include a claim code and a disclosed specialty. The processor 112 can generate a query code utilization profile 400 for the healthcare provider of the query healthcare claim 602. The processor 112 can determine a predicted healthcare provider specialty for the query healthcare claim 602 by applying the query code utilization profile 400 to a predictive model 510 generated for predicting a healthcare provider specialty. The processor 112 can determine whether a behavior of the healthcare provider of the query healthcare claim data is anomalous based on the predicted healthcare provider specialty. The processor 112 can assess the query healthcare claim for fraud, waste, or abuse based on the behavior of the healthcare provider.

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein.

It should be noted that terms of degree such as “substantially”, “about” and “approximately” when used herein mean a reasonable amount of deviation of the modified term such that the end result is not significantly changed. These terms of degree should be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that the term “coupled” used herein indicates that two elements can be directly coupled to one another or coupled to one another through one or more intermediate elements.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example and without limitation, the programmable computers (referred to below as computing devices) may be a server, network appliance, embedded device, computer expansion module, a personal computer, laptop, personal data assistant, cellular telephone, smart-phone device, tablet computer, a wireless device or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

Various embodiments have been described herein by way of example only. Various modification and variations may be made to these example embodiments without departing from the spirit and scope of the invention, which is limited only by the appended claims. Also, in the various user interfaces illustrated in the drawings, it will be understood that the illustrated user interface text and controls are provided as examples only and are not meant to be limiting. Other suitable user interface elements may be possible.

We claim:

1. A computer-implemented method of generating a predictive model for predicting healthcare provider specialties, the method comprising operating at least one processor to:
receive historical healthcare claim data, each healthcare claim comprising a claim code, a healthcare provider, and a disclosed specialty;
generate a code utilization profile for each healthcare provider based on the historical healthcare claim data;
receive registry data comprising registry specialties for each healthcare provider;
select a training dataset comprising the code utilization profiles and corresponding registry specialties; and
train the predictive model with the training dataset to predict a healthcare provider specialty for a healthcare claim.

2. The method of claim 1, wherein operating the at least one processor to select a training dataset comprising the code utilization profiles and corresponding registry specialties comprises operating the at least one processor to, for each healthcare provider:
identify a registry specialty of the registry data for the healthcare provider;
generate a specialty correspondence indicator representative of a correspondence between the registry specialty of the registry data and the disclosed specialty of the historical healthcare claim data for the healthcare provider; and
determine whether to include, in the training dataset, the code utilization profile and corresponding registry specialty for the healthcare provider based on the specialty correspondence indicator.

3. The method of claim 2, wherein operating the at least one processor to generate a specialty correspondence indicator is based on one or more natural language processing fuzzy algorithms.

4. The method of claim 2, wherein operating the at least one processor to generate a specialty correspondence indicator representative of a correspondence between the registry specialty of the registry data and the disclosed specialty of the historical healthcare claim data for the healthcare provider comprises operating the at least one processor to:
generate at least one preliminary specialty correspondence indicator for the registry specialty and the disclosed specialty; and
obtain the specialty correspondence indicator based on the at least one preliminary specialty correspondence indicator.

5. The method of claim 4, wherein:
the at least one preliminary specialty correspondence indicator comprises a plurality of preliminary specialty correspondence indicators; and
the specialty correspondence indicator is an average of the plurality of preliminary specialty correspondence indicators.

6. The method of claim 5, wherein operating the at least one processor to determine whether to include, in the training dataset, the code utilization profile and corresponding registry specialty for the healthcare provider based on the specialty correspondence indicator comprises operating the at least one processor to exclude, from the training dataset, the code utilization profile and corresponding registry specialty for the healthcare provider if:
the specialty correspondence indicator is less than a pre-determined threshold value for the specialty correspondence indicator; or
one or more preliminary specialty correspondence indicators of the plurality of preliminary specialty correspondence indicators is less than a pre-determined threshold value for that preliminary specialty correspondence indicator.

7. The method of claim 4, wherein the at least one preliminary specialty correspondence indicator comprises at least one of:
a partial score, the partial score being based on one or more abbreviations in the registry specialty or one or more abbreviations in the disclosed specialty;
a token score, the token score being based on at least one token word of the registry specialty or at least one token word of the disclosed specialty; or
a weighted score, the weighted score being based on a length of the registry specialty and a length of the disclosed specialty.

8. The method of claim 7, wherein the token score is based on a ratio of a set of token words of the registry specialty and a set of token words of the disclosed specialty.

9. The method of claim 7, wherein the weighted score is the partial score if the length of the registry specialty is significantly longer or shorter than the length of the disclosed specialty.

10. The method of claim 1, wherein operating the at least one processor to generate a code utilization profile for each healthcare provider based on the historical healthcare claim data comprises operating the at least one processor to, for each healthcare provider:
identify healthcare claims corresponding to the healthcare provider;
determine a total number of healthcare claims corresponding to the healthcare provider;
for each healthcare claim code, determine a number of healthcare claims corresponding to the healthcare provider; and
for each healthcare claim code, determine a utilization percentage based on the number of healthcare claims corresponding to the healthcare provider for the healthcare claim code to the total number of healthcare claims corresponding to the healthcare provider.

11. The method of claim 1, wherein operating the at least one processor to select a training dataset comprising the code utilization profiles and corresponding registry specialty comprises operating the at least one processor to:
for each code utilization profile and corresponding registry specialty, determine a volume size of the healthcare provider, the volume size being one of small, average, or large;
if the healthcare provider is one of small or large volume size, exclude the code utilization profile and corresponding registry specialty from the training dataset; and
if the healthcare provider is average, include the code utilization profile and corresponding registry specialty in the training dataset.

12. The method of claim 11, wherein the healthcare provider volume size being small, average or large is based on at least one of a number of healthcare claims associated with the healthcare provider or a number of patients having healthcare claims associated with the healthcare provider.

13. The method of claim 1, wherein the healthcare provider specialty is based on a taxonomy different from a taxonomy of the disclosed specialty and a taxonomy of the registry specialty.

14. The method of claim 13, wherein the taxonomy of the healthcare provider specialty comprises a classification that corresponds to a plurality of classifications from the taxonomy of the disclosed specialty or the taxonomy of the registry specialty.

15. The method of claim 1, wherein operating the at least one processor to train the predictive model with the training dataset to predict a healthcare provider specialty for a healthcare claim comprises operating the at least one processor to reduce the training dataset dimensionality.

16. The method of claim 14, wherein the operating the at least one processor to reduce the training dataset dimensionality comprises operating the at least one processor to bicluster the training dataset.

17. The method of claim 16, wherein operating the at least one processor to bicluster the training dataset comprises operating the at least one processor to assign each healthcare claim to at least one of a claim type cluster grouping and a business code cluster grouping.

18. The method of claim 14, wherein operating the at least one processor to reduce the training dataset dimensionality comprises operating the at least one processor to apply recursive feature elimination to the training dataset.

19. The method of claim 1, comprises operating the at least one processor to compare the healthcare provider specialty predicted by the predictive model to one or more pre-determined business rules.

20. A system for generating a predictive model for predicting healthcare provider specialties, the system comprising at least one processor configured to:
receive historical healthcare claim data, each healthcare claim comprising a claim code, a healthcare provider, and a disclosed specialty;
generate a code utilization profile for each healthcare provider based on the historical healthcare claim data;
receive registry data comprising registry specialties for each healthcare provider;
select a training dataset comprising the code utilization profiles and corresponding registry specialties; and
train the predictive model with the training dataset to predict a healthcare provider specialty for a healthcare claim.
